(19) United States
(12) Patent Application Publication
(10) Pub. No.: US 2025/0132046 A1
(43) Pub. Date: Apr. 24, 2025

(54) HEALTH INDEX MAINTENANCE/MANAGEMENT DEVICE FOR ARTIFICIAL INTELLIGENCE MODEL AND SYSTEM COMPRISING SAME

(71) Applicant: AINATION CO., LTD., Seoul (KR)

(72) Inventors: Jihoon Kwak, Gyeonggi-do (KR); Sangeun Lee, Seoul (KR)

(73) Assignee: AINATION CO., LTD., Seoul (KR)

(21) Appl. No.: 18/691,785

(22) PCT Filed: Sep. 17, 2021
(86) PCT No.: PCT/KR2022/013774

§ 371 Date: Mar. 13, 2024

(30) Foreign Application Priority Data
Sep. 17, 2021 (KR) 10-2021-0125303
Sep. 17, 2021 (KR) 10-2021-0125304
Sep. 17, 2021 (KR) 10-2021-0125306
Sep. 17, 2021 (KR) 10-2021-0125309

(51) Int. Cl. G16H 50/30

(52) U.S. Cl. CPC G16H 50/30

(57) ABSTRACT

A health index maintenance and management system for an artificial intelligence model is for determining the health index of an artificial intelligence model receiving input data so as to classify same into any one of a plurality of classes, and including: a learning device for generating a trained model for predicting the class of input data; a prediction device which receives the trained model generated by the learning device, and which receives input data so as to perform prediction in order to classify the class of the input data; and an operation device which receives the input data and a prediction value from the prediction device, and which calculates the confidence of the input data from the prediction value so as to determine the health index of the trained model used by the prediction device.

### TECHNICAL FIELD

The present invention relates to a device for determining and managing a health index of an artificial intelligence (AI) model so that the health index is maintained at a certain level or higher and a system including the device.

### BACKGROUND ART

The content described below simply provides background information only related to the present embodiment and does not constitute the conventional art.

With the advent of deep learning, machine learning, or artificial intelligence (AI), the corresponding technology is being applied not only to simple and repetitive tasks but also to fields that require human judgment and is replacing human processing.

In general, according to the corresponding technology, input data labeled by humans is input and learned in a specific deep learning model architecture so that relationships between input data and labels are accumulated. For example, it is assumed that a deep learning, machine learning, or AI device instead of a human performs a task of classifying images with circles and images with quadrangles. In this case, the classification device receives images that are classified as a circle or a quadrangle, and a specific architecture learns the images to generate a trained model. After that, when an image with any one of a circle or a quadrangle is input, the classification device determines whether a circle or a quadrangle is in the image using the trained model and classifies the input image as a specific class (circle or quadrangle).

In this way, the trained model that has been generated once through training is stored in the corresponding device and infers into which class input (real time) data is to be classified.

However, problems appear when a class of input data varies from a class at the time that the trained model is generated or when the boundary between classes is ambiguous. According to the foregoing example, the input data at the time that the corresponding device generates the trained model includes only two classes, circle and quadrangle. However, data with shapes which are slightly modified from the existing class data may be generated over time, data that exists at the boundary between the classes and thus is difficult to classify or requires human labeling may be generated, or data of another class that is totally different from the existing classes may be generated. For example, input data with a circle may be slightly and gradually modified so that input data with an oval may be generated. Alternatively, data of a pentagon which is at the boundary between the existing classes (circle and quadrangle) may be generated, or a class that is completely irrespective of the existing classes (e.g., a star shape) may be generated.

Even when input data of the foregoing class is input, a trained model which is generated in advance can classify the input data into only a class that has been learned. Accordingly, data of a new class is classified into a class that is not the correct answer, which lowers a correct answer rate. Meanwhile, in the case of data slightly modified from data of an existing class or data existing at the boundary between classes, even when the corresponding device classifies the data into any one class, it is uncertain whether the class is the correct answer or whether the data has been classified after correct recognition. In other words, confidence of the classification is not ensured.

For this reason, there is a demand for a method of recognizing and addressing the problem of confidence degradation in classifications of the corresponding device and a lowered correct answer rate that may result from the confidence degradation.

### DISCLOSURE

### Technical Problem

The present invention is directed to providing a device for determining and managing a health index of an artificial intelligence (AI) model so that the health index is maintained at a certain level or higher and a system including the device.

### Technical Solution

One aspect of the present invention provides a system for determining a health index of an AI model which receives and classifies input data into any one of a plurality of classes, the system including a training device configured to generate a trained model for inferring a class of input data, an inference device configured to receive the trained model generated by the training device, receive input data, and make an inference for classifying the input data into a class, and an operation device configured to receive the input data and an inference value from the inference device, calculate a confidence of the input data from the inference value, and determine a health index of the trained model used by the inference device.

The trained model may be trained using labeled input data, architectures, a cost function, and an optimization method.

The trained model may make the inference for classifying the input data into a class and also cluster data which is inferred as the same class in a latent space.

A cost value determined by the cost function may be a value proportional to a difference between the inference value of the input data and a result value of the input data.

A cost value determined by the cost function may be a value that is proportional to a difference between the inference value of the input data and a result value of the input data and proportional to a distance between the inference value of the input data and other data classified into the same class as the inference value of the input data.

The confidence may be a level of confidence that the input data will be classified into a specific class when the inference device makes an inference for classifying the input data into the specific class.

The confidence may be calculated as a distance between each class and an inference value in a latent space.

The confidence may be calculated as a ratio of a distance between each class and an inference value in a latent space.

Another aspect of the present invention provides a method in which a system for determining a health index of an AI model including a training device, an inference device, and an operation device receives and classifies input data into any one of a plurality of classes, the method including a generation process in which the training device generates a trained model for inferring a class of input data, a first receiving process in which the inference device receives the trained model from the training device and receives input data, an inference process in which the inference device makes an inference for classifying the input data into a class, a second receiving process in which the operation device receives input data and an inference value generated in real time from the inference device, and a determination process in which the operation device calculates a confidence of the input data from the inference value and determines a health index of the trained model used by the inference device.

The trained model may be trained using labeled input data, an architecture, a cost function, and an optimization method.

The trained model may make the inference for classifying the input data into a class and also cluster data which is inferred as the same class in a latent space.

A cost value determined by the cost function may be a value proportional to a difference between the inference value of the input data and a result value of the input data.

A cost value determined by the cost function may be a value that is proportional to a difference between the inference value of the input data and a result value of the input data and proportional to a distance between the inference value of the input data and other data classified into the same class as the inference value of the input data.

The confidence may be a level of confidence that the input data will be classified into a specific class when the inference device makes an inference for classifying the input data into the specific class.

The confidence may be calculated as a distance between each class and an inference value in a latent space.

The confidence may be calculated as a ratio of a distance between each class and an inference value in a latent space.

### Advantageous Effects

As described above, according to an aspect of the present embodiment, it is possible to determine the confidence of an artificial intelligence (AI) model by determining a health index of the AI model.

Also, according to an aspect of the present embodiment, it is possible to maintain and manage a health index of an AI model according to a determination result of the health index of the AI model.

### DESCRIPTION OF DRAWINGS

FIGS. 1A to 1D are diagrams showing a configuration of a system for managing a health index of an artificial intelligence (AI) model according to an embodiment of the present invention.

FIG. 2 is a diagram showing a configuration of a training device according to an embodiment of the present invention.

FIG. 3 is a diagram illustrating a class-based clustering method of a training device according to an embodiment of the present invention.

FIGS. 4A to 4C are graphs illustrating classes clustered by class by a training device according to an embodiment of the present invention.

FIG. 5 is a diagram showing a configuration of an inference device according to a first embodiment of the present invention.

FIG. 6 is a diagram showing a configuration of an operation device according to the first embodiment of the present invention.

FIG. 7 is a diagram showing a relationship between each class classified by an operation device according to an embodiment of the present invention and an inferred inference value in a latent space.

FIG. 8 is a diagram showing an arrangement of classes classified by an operation device according to an embodiment of the present invention in a latent space.

FIG. 9 is a timing chart illustrating a method in which the operation device according to the first embodiment of the present invention determines the health index of a trained model.

FIG. 10 is a timing chart illustrating a method in which the operation device according to the first embodiment of the present invention selects data for training or retraining.

FIG. 11 is a timing chart illustrating a method in which the operation device according to the first embodiment of the present invention selects and outputs a sample together with an inference value of input data.

FIG. 12 is a flowchart illustrating a method in which the operation device according to the first embodiment of the present invention determines the health index of a trained model.

FIG. 13 is a flowchart illustrating a method in which the operation device according to the first embodiment of the present invention selects data for training or retraining.

FIG. 14 is a flowchart illustrating a method in which the operation device according to the first embodiment of the present invention selects and outputs a sample together with an inference value of input data.

FIG. 15 is a diagram showing a configuration of an inference device according to a second embodiment of the present invention.

FIG. 16 is a diagram showing a configuration of an operation device according to the second embodiment of the present invention.

FIG. 17 is a timing chart illustrating a method in which the operation device according to the second embodiment of the present invention determines the health index of a trained model.

FIG. 18 is a timing chart illustrating a method in which the operation device according to the second embodiment of the present invention selects data for training or retraining.

FIG. 19 is a timing chart illustrating a method in which the operation device according to the second embodiment of the present invention selects and outputs a sample together with an inference value of input data.

FIG. 20 is a flowchart illustrating a method in which the operation device according to the second embodiment of the present invention determines the health index of a trained model.

FIG. 21 is a flowchart illustrating a method in which the operation device according to the second embodiment of the present invention selects data for training or retraining.

FIG. 22 is a flowchart illustrating a method in which the operation device according to the second embodiment of the present invention selects and outputs a sample together with an inference value of input data.

FIG. 23 is a timing chart illustrating a training method of a training device according to an embodiment of the present invention.

FIG. 24 is a timing chart illustrating a method in which a system for managing a health index of an AI model according to an embodiment of the present invention determines the confidence of a trained model.

### MODES OF THE INVENTION

Since the present invention can be variously modified and have several embodiments, specific embodiments will be illustrated in the drawings and described in detail below. However, the embodiments are not intended to limit the present invention to specific forms of implementation, and it should be understood that the present invention includes all modifications, equivalents, and substitutions within the spirit and technical scope of the present invention. Throughout the drawings, like reference numerals refer to like components.

The terms “first,” “second,” “A,” “B,” and the like may be used for describing various components, but the components are not limited by the terms. The terms are only used for the purpose of distinguishing one component from another. For example, a first component may be named a second component without departing from the scope of the present invention, and a second component may likewise be named a first component. The term “and/or” includes any one or a combination of a plurality of related stated items.

When a first component is referred to as being “connected” or “coupled” to a second component, the first component may be directly connected or coupled to the second component, or a third component may be therebetween. On the other hand, when a component is referred to as being “directly connected” or “directly coupled” to another component, there is no intermediate component therebetween.

Terminology used in this specification is used only for describing specific embodiments and is not intended to limit the present invention. The singular forms include the plural forms as well unless the context clearly indicates otherwise. In this specification, the terms “comprise,” “include,” “have,” and the like do not preclude the possibility of presence or addition of one or more features, integers, steps, operations, components, parts, or combinations thereof stated herein.

Unless otherwise defined, all terms including technical or scientific terms used herein have the same meaning as generally understood by those of ordinary skill in the art.

Terms defined in generally used dictionaries are construed as having the same meaning as would be construed in the context of the related art. Unless defined clearly and apparently in this specification, the terms are not construed as having ideal or excessively formal meanings.

In addition, each element, operation, process, method, or the like included in each embodiment of the present invention may be shared unless technically contradictory to another.

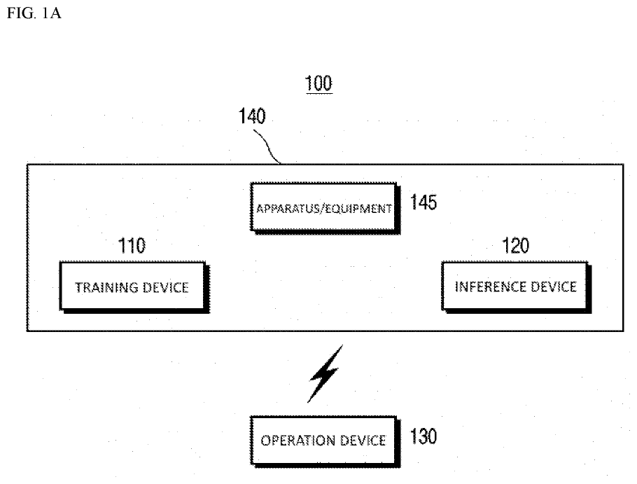

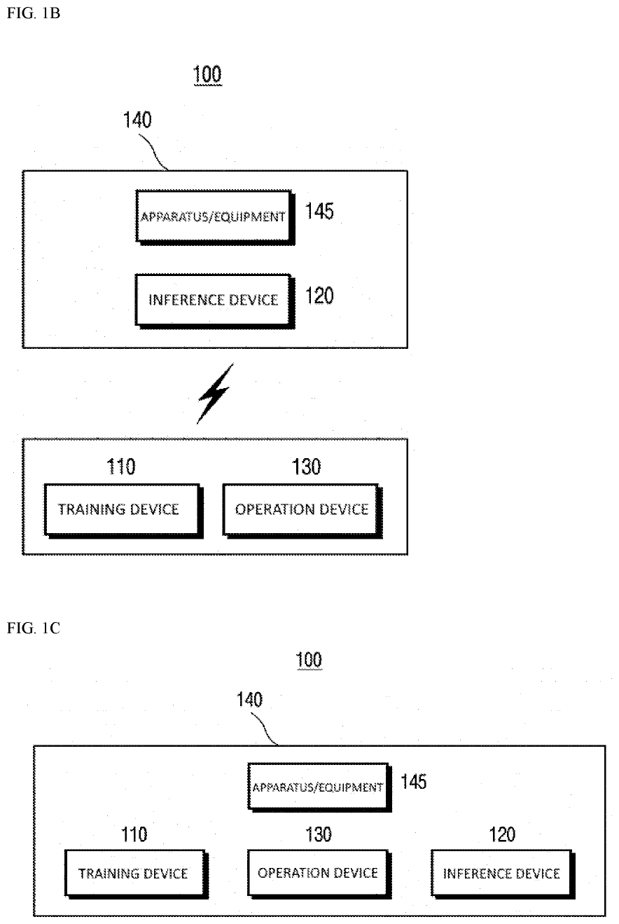

FIGS. 1A to 1D are diagrams showing a configuration of a system for managing a health index of an artificial intelligence (AI) model according to an embodiment of the present invention.

Referring to FIGS. 1A to 1D, a system 100 for managing a health index of an AI model according to an embodiment of the present invention includes a training device 110, an inference device 120, and an operation device 130. It will be described below that data or the like is directly transmitted from a specific device (e.g., the training device 110) and received by another device (e.g., the inference device 120), but the present invention is not necessarily limited thereto. Data or the like may be transmitted from a specific device and received by another device through an additional medium such as a database, a Universal Serial Bus (USB) device, or the like. For convenience, however, it will be described below that data or the like is directly transmitted and received.

An apparatus/equipment 145 is disposed in a certain space 140 and outputs data that requires human judgment or examination. For example, the space 140 may be a factory, and the apparatus/equipment 145 may be manufacturing equipment for producing a specific material, part, or the like. One or more apparatuses/equipment 145 may be disposed in the space 140 and output data requiring human judgment or examination. Data generated in advance by the apparatus/equipment 145 is provided to the training device 110 so that the training device 110 may generate a trained model by training a model. Meanwhile, data generated in real time by the apparatus/equipment 145 is provided to the inference device 120 so that the inference device 120 may infer a specific class from the input data using the trained model.

A manager or operator of the space 140 or a person concerned with data output from the apparatus/equipment 145 (hereinafter, “person concerned”) may check an inference value inferred by the inference device 120 using the generated trained model. Also, the person concerned may indirectly determine whether the trained model makes a correct inference from data which is input in real time by checking a health index of the trained model which is output by the operation device 130. Here, the health index of the trained model indicates how well an inference value inferred by the trained model corresponds to a correct answer, and may indicate the accuracy (correct answer rate) of an inference value of the trained model. The person concerned may check the health index of the trained model directly from the inference device 120 or the operation device 130 or by communicating with the inference device 120 or the operation device 130 through his or her terminal (not shown).

In addition, a person who may determine or examine a result himself or herself on the basis of data (e.g., a labeler) (hereinafter, “labeler”) may label or relabel data which is selected by the operation device 130 for training or retraining. When the training device 110 performs training or retraining using data which is labeled or relabeled by the labeler, the trained model may make an inference from input data with higher accuracy.

The training device 110 receives a dataset for training, a deep learning model architecture (hereinafter, “architecture”), a cost function, and an optimization method from a terminal (not shown) of the person concerned and generates a trained model. As the dataset for training, the training device 110 receives input data and result values with which the input data is labeled, and also receives the architecture, the cost function, and the optimization method. Here, the cost function is defined as follows. A first cost function may be or include a function that is proportional to the distance between an inference value and other data of the same class as of the inference value in the latent space. A second cost function may be a function that is proportional to the distance between an inference value and the result value (correct answer value) or a function that is proportional to the distance between an inference value and other data of the same class as of the inference value in the latent space, or may include one or both of the two functions. A cost value indicates a value derived through the cost function. An inference value is calculated per class as a possibility value indicating that the input data will correspond to the class, and a result value is the result of which class input data corresponds to. A result value has a 100% possibility only for the corresponding class and has 0% possibility for other classes. For example, when there are three classes and input data is to be classified into a first class, inference values may be (0.7, 0.2, 0.1) (the values are variable according to confidence), and the result values may be (1, 0, 0). In this way, the difference between an inference value and the result value may be calculated, and a cost value (of the second cost function) indicates a value that is proportional to the difference between an inference value and the result value (correct answer value). Meanwhile, the first cost function may be a function that is proportional to the distance between an inference value and other data of the same class as of the inference value in the latent space. The optimization method is a method of minimizing a cost value determined through the cost function. The training device 110 receives the foregoing data and performs training by continuously calculating and selecting parameters in the architecture in the direction of minimizing the cost value. Accordingly, as a trained model, the training device 110 generates an architecture with parameters that are set to minimize the cost value. When the cost function is defined as the first cost function in the trained model generated by the training device 110, the trained model makes an inference so that the distance between an inference value and other data of the same class as of the inference value in the latent space may be minimized. On the other hand, when the cost function is defined as a function that is proportional to the difference between an inference value and the result value (correct answer value) among the second cost functions in the trained model generated by the training device 110, the trained model makes an inference so that input data may be classified into a class having the minimized difference with a correct answer value. The training device 110 generates a trained model that may perform the foregoing operations on the basis of a received input.

Here, data or an inference value disposed in the latent space has a matrix or a vector value derived from the matrix. Accordingly, the training device 110 may calculate parameters for minimizing a cost value according to the optimization method and generate a trained model.

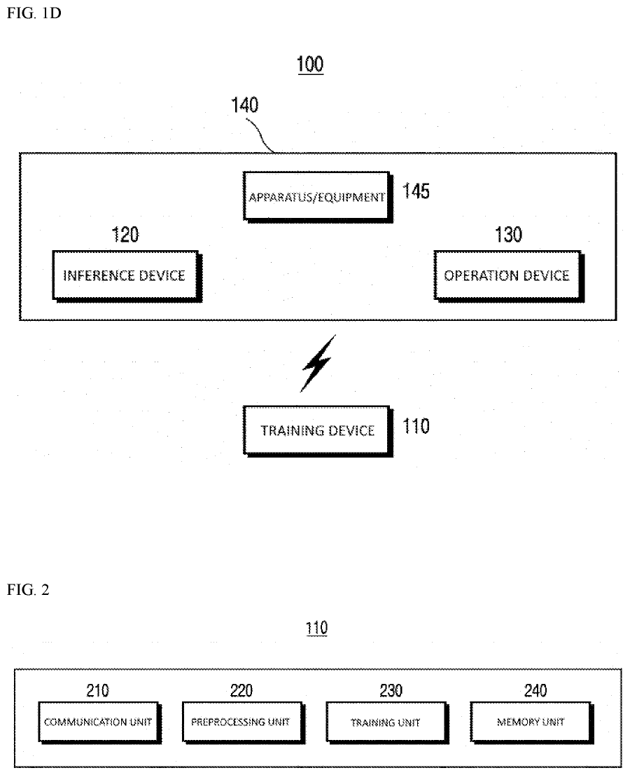

The training device 110 receives data selected for (re) training from the operation device 130 and generates a trained model or retrains a previously generated trained model. In general, when a larger amount of (labeled) input data is input for training, the inference accuracy of a trained model increases. However, the inference accuracy of a trained model is not necessarily proportional to the amount of input data. Since humans label data one by one, labeled input data may include the following data. Data without any problem may be labeled wrong, and data which is noise may be labeled and included in input data. When such data is included in the input data, a trained model shows low accuracy. Also, when there is a considerable amount (e.g., tens of thousands, hundreds of thousands, or more) of training data, even if the data has a high error rate, a trained model may recognize that corresponding data is errors on the basis of data learned from other data. However, when there is a small amount (e.g., thousands or less) of training data and there are errors in the data, the errors may have critical effects on inference. In addition, when there is a considerable amount of data and the proportion of relatively low-importance data to relatively high-importance data is very high, learning of the relatively high-importance data does not proceed smoothly, which lowers the inference accuracy of a trained model. As described in the example of Background Art, in a model for classifying circles and quadrangles, input data that exists at the boundary between circular shapes and quadrangular shapes and thus is difficult to classify into any one may be more important than input data that is clearly circle-shaped and quadrangle-shaped. When a considerable number of pieces of such data are learned, a relatively accurate inference may be made even from input data existing at the boundary. However, as described above, when the proportion of relatively low-importance data is much higher than that of relatively high-importance data, there is a high possibility that learning of the relatively high-importance data will not proceed smoothly. Therefore, a large number of datasets to be learned does not ensure the inference accuracy of a trained model. In this regard, the training device can ensure and maintain the inference accuracy of the trained model by performing training or retraining using input data that is selected by the operation device 130 and labeled by the labeler. A detailed configuration of the trained model will be described below with reference to FIG. 2.

The inference device 120 receives the trained model generated by the training device 110 and makes an inference to classify data generated in real time by the apparatus/equipment 145 into a class. In the case of making an inference from input data using the trained model, the inference device 120 does not output an inference value as a class but calculates a possibility that the inference value will correspond to each class. A result may indicate an absolutely high possibility that the inference value will correspond to a specific class, or indicate that the inference value will correspond to a specific class by a slight (possibility) margin. The inference device 120 may make an inference from a possibility value that input data will correspond to a certain class using the trained model. The inference device 120 receives and stores the trained model which is generated by the training device 110 through the foregoing process, and receives data which is generated in real time by the apparatus/equipment 145. The inference device 120 classifies input data which is received real-time data into a class using the trained model.

The inference device 120 stores input data which is received for a preset period of time and inference values of the input data and transmits the input data and the inference values to the operation device 130.

The operation device 130 receives the input data and the inference values of the input data from the inference device 120, calculates the confidences of the inference values, and determines the health index of the trained model stored in the inference device 120 or selects data for training or retraining on the basis of the confidences.

The operation device 130 receives the input data and the inference values of the input data from the inference device 120 and calculates the confidences of the inference values on the basis of the received values. A confidence may be a level of confidence that input data will be classified into a specific class in the case of making an inference to classify the input data into the specific class. A confidence may be calculated as the distance between each class (more specifically, the center of each class) (hereinafter, this may mean “the center of each class” even if not specifically mentioned) and an inference value in the latent space, more specifically, a distance ratio between each class and an inference value. Here, the distance may be a Euclidian distance but is not necessarily limited thereto. An inference value with a high confidence has a short distance between a specific class and the inference value (classified into the specific class) but long distances between other classes and the inference value (only the distance from the class into which the inference value is classified is short). Distances between an inference value with a low confidence and all classes are a preset reference value or more, or distances between the inference value with the low confidence and two or more classes are similar (distances from not only the class into which the inference value is classified but also other classes are short or long). When the number of inference values with a low confidence increases, this may mean that data of a new class or data at the boundary between two or more classes is appearing or increasing. Accordingly, the operation device 130 determines that a correct answer rate is decreasing or is likely to decrease due to the health index of the trained model being lowered. The operation device 130 directly outputs a notification to the person concerned that the heal index of the trained model has decreased or a notification to the terminal (not shown) of the person concerned that it is necessary to retrain the trained model.

The operation device 130 selects data for training or retraining on the basis of the calculated confidences. The operation device 130 may calculate the confidence of each inference value and classify the feature of input data having the inference value. Input data is classified by feature as follows. Inference data may be classified into data of which an inference value is located within the radius of a specific class in the latent space and which may thus be fully classified into a specific class only (hereinafter, “first group”), data of which an inference value is located outside the radii of all classes in the latent space but which is located relatively adjacent to two or more classes (at a distance of the preset reference value or less) (hereinafter, “second group”), and data of which an inference value is at a distance of the preset reference value or more from all the classes in the latent space (hereinafter, “third group”). To classify features of data, information on the distance from a specific class in all latent spaces is required, and thus it is necessary to calculate a confidence. The operation device 130 calculates a confidence from the inference value of each piece of input data and determines to which group a feature of the input data corresponds on the basis of the confidence. In this way, the operation device 130 classifies each piece of input data by feature and a preset number of pieces of data per feature. As described above, factors that reduce inference accuracy of data which is input in real time are data belonging to the second group or input data of a new class in the third group. Since it is necessary to mainly learn such input data, the operation device 130 selects the preset number of pieces of data per feature so that data of the second group and data of the third group may be included at a certain ratio. For example, the operation device 130 may select data from the first, second, and third groups at a ratio of 4:3:3, 6:2:2, or the like, respectively. The operation device 130 transmits the selected data to the training device 110 so that the training device 110 can initially train a model using only the selected data or retrain a model using the selected data. As described above, even when there is a large amount of training data, the correct answer rate of a trained model does not increase unconditionally. Therefore, the operation device 130 may increase the correct answer rate of a trained model to be initially generated by selecting data or increase the correct answer rate of a trained model by performing retraining when input data (which is generated in real time) is changed.

The operation device 130 may directly output input data which is classified into the second group or the third group so that the labeler may smoothly label the data. In the case of outputting the input data, the operation device 130 may also output an example of some of data (with a high confidence) previously classified into each class that has a slight possibility difference in the inference process of the input data. Alternatively, the operation device 130 may also output an example of some of data previously classified into the same feature as input data. For example, when input data with a low confidence corresponds to the second group, the operation device 130 may also output some of data previously classified into each class that has a slight possibility difference in the inference process of the input data or may also output an example of some of various data classified into the second group. In particular, when the operation device 130 outputs data classified into the same feature, the labeler can know into which class data that has been classified into the second group is classified, which may help with classifying corresponding input data on the basis of the same criteria as before.

Schematic operations of the system 100 for managing a health index of an AI model are as follows.

Each piece of data which is input and classified during class determination is stored in the inference device 120 and the operation device 130 together with its confidence.

While classifying data into a class, the operation device 130 continuously monitors the health index, and at a point in time when the health index is at a certain level or lower, notifies the person concerned, the terminal carried by the person concerned, or the like of that.

The operation device 130 selects data that is determined to show a high training effect for restoring the health index from among the stored data on the basis of confidences, and the person concerned labels the selected data with the help (labeling guidance) of the operation device 130.

In this way, the system 100 for managing a health index of an AI model selects data, which is difficult to classify on the basis of previous training and thus causes a decrease in the health index, and performs training or retraining with the selected data. Accordingly, it is possible to maintain the health index of the model at a certain level.

On the other hand, to maintain a health index according to the conventional art, the person concerned should randomly select a certain ratio of data classification results, find correct answer values for the data classifications one by one, and monitor how closely the data classifications correspond to inference values at all times. This involves a certain number of people at all times.

When the system 100 for managing a health index of an AI model according to an embodiment of the present invention is used, the person concerned can monitor the health index at all times and easily find a point in time when the health index decreases to a preset level or lower.

Also, according to the conventional art, additional data for retraining is randomly selected at a certain ratio, and thus it is highly likely that a large number of pieces of data which are determined in advance to have high confidences and are not efficient for additional training will be included. Therefore, according to the conventional art, a relatively large number of pieces of training data are required for restoring the health index through retraining.

On the other hand, the system 100 for managing a health index of an AI model according to an embodiment of the present invention can perform retraining using only a (relatively) small amount of data that is efficiently selected on the basis of confidences (after being labeled with the help of the operation device 130). Therefore, a small number of people can maintain the health index of a data classification device at a preset level or higher in only a minimum period of time.

Each of the training device 110, the inference device 120, and the operation device 130 may be disposed as shown in any one of FIGS. 1A to 1D.

As shown in FIG. 1A, both the training device 110 and the inference device 120 may be disposed in the space 140 together with the apparatus/equipment 145. The training device 110 may receive training data directly from the apparatus/equipment 145 and generate a trained model, and the inference device 120 may infer data generated in real time by the apparatus/equipment 145 using the trained model.

The operation device 130 may transmit or receive required data by communicating with each device 110, 120, or 145. The operation device 130 may receive input data and an inference value from the inference device 120 and notify the training device 110 or the person concerned of a health index of the trained model and selected data.

The person concerned may check the inference value in real time using the inference device 120, and the person concerned or the labeler may determine the health index of the trained model using the operation device 130 regardless of location.

The training device 110 may receive data from the apparatus/equipment 145 through wired communication. The training device 110 may be implemented in the form of edge computing or the like and receive data directly from the apparatus/equipment 145.

As shown in FIG. 1B, only the inference device 120 may be disposed in the space 140 to make an inference, and the training device 110 and the operation device 130 at a separate location may transmit or receive required data to or from the apparatus/equipment 145 or the inference device 120.

In this case, the transmitted or received data, particularly, data transmitted from the apparatus/equipment 145 to the training device 110, may be encrypted. Data that is output from the apparatus/equipment 145 or output from the apparatus/equipment 145 and accumulated may correspond to important assets for a specific industry and thus may necessarily require security. For this reason, data to be transmitted by the apparatus/equipment 145 may be encrypted and transmitted, and the training device 110 may generate a trained model by receiving and decrypting the data.

As shown in FIG. 1C, all the devices 110 to 130 of the system 100 for managing a health index of an AI model may be disposed in the space 140 and operate.

Otherwise, as shown in FIG. 1D, the inference device 120 and the operation device 130 may be disposed in the space 140, and the training device 110 may be disposed in a separate location.

However, the present invention is not necessarily limited thereto, and the devices 110 to 130 may be disposed in any arrangement in relation to the space 140 as along as each of the devices 110 to 130 can perform the foregoing operations.

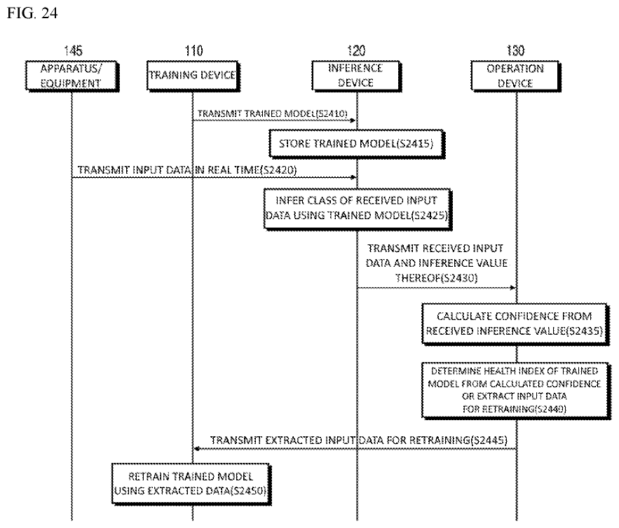

A schematic operation of each of the devices 110 to 130 of the system 100 for managing a health index of an AI model is illustrated in FIG. 24.

FIG. 24 is a timing chart illustrating a method in which a system for managing a health index of an AI model according to an embodiment of the present invention determines the confidence of a trained model.

The training device 110 generates and transmits a trained model to the inference device 120 (S2410).

The inference device 120 stores the received trained model (S2415).

The inference device 120 receives input data from the apparatus/equipment 145 in real time (S2420).

The inference device 120 infers a class of the received input data using the trained model (S2425).

The inference device 120 transmits the received input data and an inference value of the input data to the operation device 130 (S2430).

The operation device 130 calculates a confidence from the received inference value (S2435).

The operation device 130 determines a health index of the trained model from the calculated confidence or selects input data for retraining (S2440).

The operation device 130 may transmit the selected input data for retraining to the training device 110 (S2445).

The training device 110 may retrain the trained model using the selected data (S2450).

FIG. 2 is a diagram showing a configuration of a training device according to an embodiment of the present invention.

Referring to FIG. 2, the training device 110 according to an embodiment of the present invention includes a communication unit 210, a preprocessing unit 220, a training unit 230, and a memory unit 240.

The communication unit 210 receives labeled input data, a cost function, and an optimization method from the apparatus/equipment 145 or the terminal (not shown) of the person concerned and receives data selected for training or retraining from the operation device 130. In some cases, the communication unit 210 may further receive an architecture from the apparatus/equipment 145 or the terminal (not shown) of the person concerned.

The communication unit 210 may additionally receive labeled input data for a test from the apparatus/equipment 145 or the terminal (not shown) of the person concerned.

The preprocessing unit 220 preprocesses the received (labeled) input data or selected data so that the training unit 230 may perform training using the selected data. The labeled input data or selected data is data that the apparatus/equipment 145 outputs for its purpose, and may have a different format from a data format for training of the training unit 230. The preprocessing unit 220 converts the format of the received labeled input data or selected data so that the data can be transmitted to the training unit 230 and used for training without difficulty.

The training unit 230 trains a model (generates a trained model) using the labeled input data, the cost function, the optimization method, and the architecture. The training unit 230 itself may select the architecture of the model or externally receive the architecture. Here, a neural network may be used as the architecture, or one of various neural networks may be selected according to the form of the input data. For example, when the input data is an image, the architecture may be implemented as a convolution neural network (CNN) or the like. In the case of the input data other than images, the architecture may be implemented as a multi-layer perceptron. When a time-series element is included in the input data, the architecture may be implemented as a recurrent neural network (RNN). However, the foregoing examples are only illustrative, and the architecture may be implemented as various types according to the form of the input data.

The training unit 230 trains the selected or received architecture on the basis of the labeled input data, the cost function, and the optimization method. The training unit 230 randomly selects each parameter in the architecture and applies labeling results and the input data to the architecture (of which parameters are selected) to calculate the cost function. After that, the training unit 230 reselects parameters while changing the parameters according to the optimization method so that a cost value decreases. An example of the optimization method is gradient descent which is a method of adjusting the parameters in the direction of reducing the gradient of the cost function. Through this process, the training unit 230 trains the model by selecting parameters that result in the minimum cost value when the labeled input data is applied to the selected architecture. The trained model is generated according to this training process of the training unit 230.

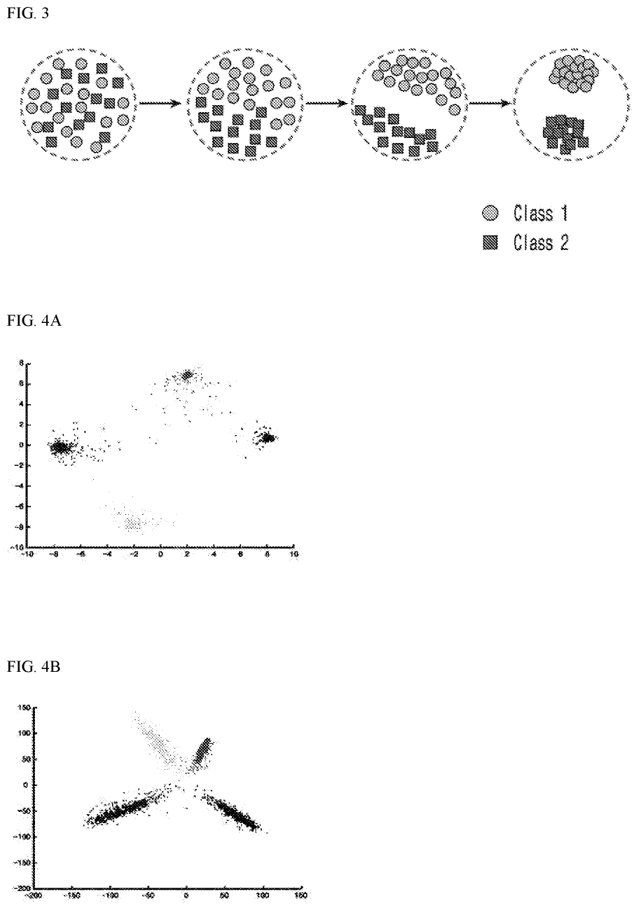

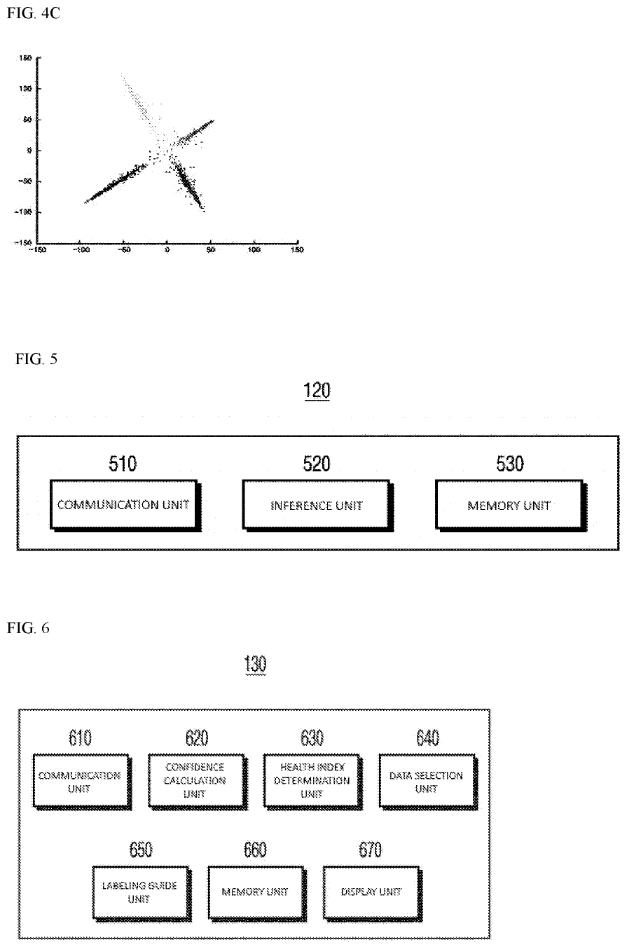

The trained model generated by the training unit 230 has a characteristic that a cost value is minimized. As described above, the cost function corresponds to a value that is proportional to the difference between an inference value and the result value (correct answer value) and/or proportional to the distance between the inference value and other data of the same class as of the inference value in the latent space. Accordingly, when input data is input, the generated trained model may make an inference as close as possible to the correct answer value of the input data (minimizing the difference in possibility between classes). In addition, the trained model makes an inference as described above according to the definition of the cost function and also places the inference value so that inference values of the same class may be clustered as much as possible in the latent space. Inference values disposed by the training unit in the latent space are shown in FIGS. 3 and 4A to 4C.

FIG. 3 is a diagram illustrating a class-based clustering method of a training device according to an embodiment of the present invention, and FIGS. 4A to 4C are graphs illustrating classes clustered by class by a training device according to an embodiment of the present invention.

As described above, data or an inference value disposed in a latent space has a matrix or a vector value derived from the matrix, and thus the number of dimensions increases according to elements of the vector. For example, when the vector has n elements, a latent space has n dimensions. FIGS. 3 and 4A to 4C show such n-dimensional latent spaces projected to two dimensions.

FIG. 3 shows an example in which there are two classes. Referring to FIG. 3, when a cost function is only defined as a value that is proportional to the difference between an inference value and the result value (correct answer value) among the second cost functions and input data is input to the trained model, inference values are calculated as class 1 or class 2 as shown in the leftmost diagram.

Here, when the cost function is defined as the first cost function or a value that is proportional to the distance between an inference value and other data of the same class as of the inference value, the intervals between data (inference values) of each class gradually decrease, and data of each class is clustered as shown in the rightmost diagram. The shapes of clusters are determined depending on a method of defining the cost function.

FIG. 4A is a diagram in which data of the same class is clustered, and FIGS. 4B and 4C are diagrams in which data of the same class is clustered at the same angle.

As seen from FIGS. 3 and 4A to 4C, the trained model generated by the training unit 230 may not only classify input data into classes but also cluster data of the classified classes.

Referring back to FIG. 2, the training unit 230 may test the generated trained model using labeled input data for a test. Since the labeled input data for a test is received, the training unit 230 may infer a class of the input data on the basis of the input data and compare the inference value with the label value (result value). The training unit 230 may check a correct answer rate of inferences of the trained model on the basis of the comparison result.

The training unit 230 may generate the trained model (the process of training a model) according to the above-described process using input data which is selected (labeled) by the operation device 130 for training. Noise of the input data selected by the operation device 130 may be minimized, and data of the first to third groups may be included at an appropriate ratio. Accordingly, the trained model generated by the training unit 230 can have higher accuracy than a conventional trained model which is trained using a huge number of pieces of reckless input data.

Also, the training unit 230 may retrain a previously generated trained model using input data which is selected by the operation device 130 for retraining. When input data of the second or third group increases, it is difficult to make an inference normally using a trained model which is generated using input data of the first group. Accordingly, the training unit 230 receives input data which is selected (labeled) for retraining from the operation device 130 and retrains the trained model using the selected input data according to an architecture, a cost function, and an optimization method of the generated trained model. Therefore, the training unit 230 can increase the inference accuracy (correct answer rate) of the trained model.

The memory unit 240 stores (labeled) input data received from the apparatus/equipment 145 and a trained model generated by the training unit 230. Further, the memory unit 240 may store (labeled) input data which is received from the operation device 130 and selected for training or retraining and a trained model which is retrained by the training unit 230. Since the memory unit 240 may store not only (labeled) input data and a trained model which is trained using the input data but also (labeled) input data selected for retraining and a trained model which is trained using the selected input data, it is possible to separately distribute (transmit or sell) the (labeled) input data selected for retraining and the retrained model later.

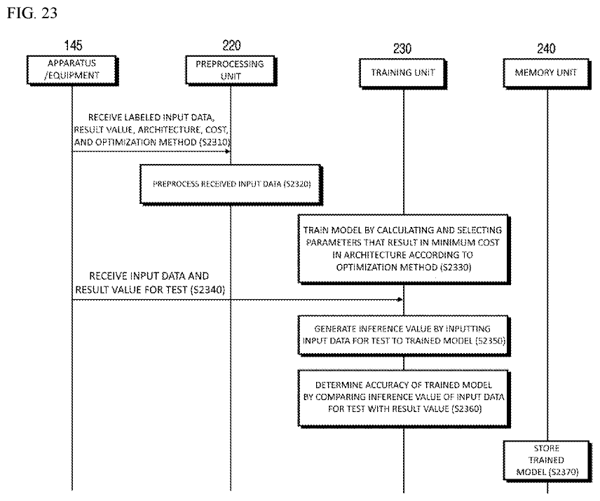

A method in which the training device 120 trains a model is shown in FIG. 23.

FIG. 23 is a timing chart illustrating a training method of a training device according to an embodiment of the present invention.

The preprocessing unit 220 receives labeled input data, an architecture, a cost function, and an optimization method from the apparatus/equipment 145 through the communication unit 210 (S2310).

The preprocessing unit 220 preprocesses the received (labeled) input data (S2320).

The training unit 230 trains a model by calculating and selecting parameters that result in the minimum cost value in the architecture according to the optimization method (S2330).

The training unit 230 receives labeled input data and output data for a test from the apparatus/equipment 145 (S2340).

The training unit 230 generates an inference value by inputting the input data for a test to the trained mode (S2350).

The training unit 230 determines accuracy of the trained model by comparing the inference value of the input data for a test with the output data (S2360).

The memory unit 240 stores the generated trained model (S2370).

In the above process, a trained model is generated, and a test is performed on the generated trained model.

FIG. 5 is a diagram showing a configuration of an inference device according to a first embodiment of the present invention.

Referring to FIG. 5, the inference device 120 according to the first embodiment of the present invention includes a communication unit 510, an inference unit 520, and a memory unit 530.

The communication unit 510 receives a trained model from the training device 110 and input data from the apparatus/equipment 240 in real time. Also, the communication unit 510 transmits an inference value to the operation device 130 together with the received input data.

The inference unit 520 makes an inference using the received trained model to classify the input data which is received in real time into a class. In the case of making an inference from the input data using the trained mode, the inference unit 520 does not infer an inference value as a class but calculates a possibility that the inference value will correspond to each class. A result may indicate an absolutely high possibility that the inference value will correspond to a specific class, or indicate that the inference value will correspond to a specific class by a slight (possibility) margin. The inference unit 520 may make an inference for class determination from a possibility value that input data will correspond to a certain class using the trained model.

The received trained model corresponds to a model that is trained to minimize a cost value of the first cost function. Accordingly, an inference value of the inference unit 520 is disposed by the trained model at an appropriate position in the latent space in which data is clustered by class.

The memory unit 530 stores the received trained model and input data and stores the inference value inferred by the inference unit 520.

FIG. 6 is a diagram showing a configuration of an operation device according to the first embodiment of the present invention.

Referring to FIG. 6, the operation device 130 according to the first embodiment of the present invention includes a communication unit 610, a confidence calculation unit 620, a health index determination unit 630, a data selection unit 640, a labeling guide unit 650, and a memory unit 660. Further, the operation device 130 may include a display unit 670.

The communication unit 610 receives input data and an inference value from the inference device 120 and transmits a health index determination result or selected data to the training device 110 or the terminal (not shown) of the person concerned. The communication unit 610 receives input data of a preset period of time and an inference value from the inference device 120 and continuously receives data and the like for a preset period of time.

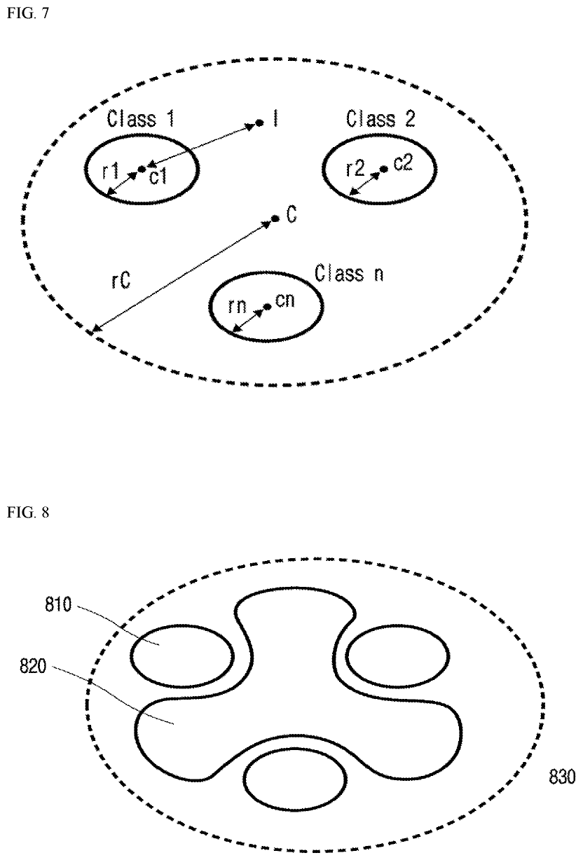

The confidence calculation unit 620 determines a confidence of each inference value using the inference value received from the inference device 120. As described above, a trained model used for inference by the inference device 120 corresponds to a model that is trained to minimize a cost value of the first cost function. Accordingly, the inference value may be disposed in the latent space, and the confidence calculation unit 620 calculates the confidence of the inference value. As described above, a confidence is calculated as the distance between each class (more specifically, the center of each class) and an inference value in the latent space, more specifically, a distance ratio between each class and an inference value. A confidence may be calculated with reference to FIGS. 7 and 8 as follows.

FIG. 7 is a diagram showing a relationship between each class classified by an operation device according to an embodiment of the present invention and an inferred inference value in a latent space, and FIG. 8 is a diagram showing an arrangement of classes classified by an operation device according to an embodiment of the present invention in a latent space.

Referring to FIG. 7, an inference value I is inferred from input data by a trained model and disposed in a latent space. In the latent space, n classes Class 1 to Class n may exist, and data classified into each class is clustered within a certain radius r₁ to rₙ. Each class has a center c₁ to cₙ. There is a center C of all the classes, and the latent space has a radius r_{c} of a preset reference value.

Data or an inference value disposed in the latent space has a matrix or a vector value derived from the matrix. Accordingly, each center may be calculated as an average value of data which is classified into the corresponding class. For example, the center c₁ to cₙ of each class may be an average value of data included in the class, and the center C of all the classes may be an average value of the centers c₁ to cₙ of the classes.

The latent space is defined as described above, and the confidence calculation unit 620 calculates a distance d₁ to dₙ between the inference value I and each class, more specifically, the center c of each class, according to a confidence calculation method.

Referring to FIG. 8, the confidence calculation unit 620 classifies a feature of input data from a calculated confidence. According to the confidence calculation, when a distance dᵢ between a center cᵢ of a specific class and an inference value d is smaller than a radius rᵢ of the specific class, the inference value corresponds to a first group 810 and has a high confidence. On the other hand, when a distance between the inference value I and the center C of all the classes is larger than the radius r_{c} (of all the classes), the inference value corresponds to a third group 830 and may have a low confidence (regardless of whether the inference value is a correct answer). When neither of the two cases described above applies, or the distance dᵢ between the center cᵢ of the specific class and the inference value d is larger than the radius r of all the classes but a distance d to the center of two or more classes is smaller than the distance to the center of another class, the inference value corresponds to a second group 820 and has a low confidence. The confidence of an inference value corresponding to the second group 820 decreases as the inference value is closer to the boundary between classes rather than the center of any one class.

Referring back to FIG. 5, the confidence calculation unit 620 calculates the confidence of each inference value inferred by the inference device 120 receiving input data as described in the above process.

The health index determination unit 630 determines the health index of the trained model using the inference value received from the inference device 120. The health index determination unit 630 determines the health index of the trained model using the confidence calculated by the confidence calculation unit 620 according to the following method.

As one method, the health index determination unit 630 calculates the number of inference values classified into the second group 820 or the average of confidences of the inference values and observes a change in the number or confidence. Since the health index determination unit 630 may calculate the foregoing data from the currently received inference value and calculate the foregoing data from previously received inference values, the health index determination unit 630 may observe a change in the number or confidence of inference values classified into the second group 820. A decrease in the average of confidences means an increase in the amount of data classified into the second group 820 and also an increase in the amount of data that is difficult to classify into a class. An increase in borderline data which is classified into the second group 820 may indicate a point in time when it is necessary to perform relabeling and retraining. Accordingly, in the above-described situation, the health index determination unit 630 may determine that the health index of the trained model has decreased (to a preset reference value or less).

As another method, the health index determination unit 630 calculates the number of inference values classified into the third group 830 or the average of confidences of the inference values and observes a change in the number or confidence. An increase in the number of pieces of data classified into the third group 830 may mean an increase in noise or an increase in the amount of data classified into a new class. Also, a sudden increase in the average of confidences compared to before may mean an increase in the amount of data classified into a new class. Accordingly, an increase in the amount of data which is classified into the third group 820 may also indicate a point in time when it is necessary to perform relabeling and retraining. Therefore, in the above-described situation, the health index determination unit 630 may determine that the health index of the trained model has decreased (to the preset reference value or less).

As another method, the health index determination unit 630 observes a change in the radius rᵢ of each class. An increase in the radius of a specific class may mean an increase in the number of pieces of data classified into a specific class. This represents that data which has been or may have been classified into the second group 820 is classified into a specific class in the latent space. On the other hand, the radius of a specific class may suddenly decrease. This represents that data which has been or may have been classified into a specific class is classified into the second group 820. Therefore, in the above-described situation, the health index determination unit 630 may determine that the health index of the trained model has decreased (to the preset reference value or less).

As still another method, the health index determination unit 630 observes a change in the coordinates of the center cᵢ of each class. A sudden change in the coordinates of the center cᵢ of each class compared to before may mean a drastic change in the operation environment of the apparatus/equipment 145 in the space 140. Such a drastic change in the environment may influence the inference accuracy of the trained model. Therefore, when the coordinates of the centers cᵢ of some or all classes are changed, the health index determination unit 630 may determine that the health index of the trained model has decreased (to the preset reference value or less).

In this way, the health index determination unit 630 uses a confidence to make various determinations and also to determine the health index of the trained model used for inference by the inference device 120. When the health index of the trained model decreases to the preset reference value or less, the health index determination unit 630 may determine that it is a point in time when it is necessary to retrain the trained model. Accordingly, the health index determination unit 630 causes the display unit 670 to display that the health index has decreased and it is a point in time when it is necessary to retrain the trained model. Also, the health index determination unit 630 may transmit this to the training device 110 or the terminal (not shown) of the person concerned through the communication unit 610.

When the health index determination unit 630 determines that it is necessary to retrain the trained model (that the health index has decreased), the data selection unit 640 selects data to be used for retraining. The data selection unit 640 selects data to be used for retraining from input data which is received from the inference device 120 and input for a preset period of time. As described above, the confidence calculation unit 620 calculates confidences and then classifies features of the received input data into classes (the first group to third group) according to the confidence of each inference value. The data selection unit 640 selects a preset ratio of data from the received input data per feature (the first group 810 to the third group 830).

The data selected by the data selection unit 640 may be selected on the basis of a different criterion for each group. The data selection unit 640 may select a certain number of pieces of data in increasing or decreasing order of confidence from the first group. The data selection unit 640 may select a certain number of pieces of data in increasing order of confidence (data disposed closer to a boundary) from the second group. The data selection unit 640 may select a certain number of pieces of data in decreasing order of distance from the center C of all the classes from the third group. The data selection unit 640 can increase the correct answer rate of the trained model to be retrained by selecting data for each feature on the basis of the foregoing criteria.

When the display unit 670 outputs data classified into the second group or the third group on the basis of its inference value, the labeling guide unit 650 selects data that may help the labeler to label the data as samples. The display unit 670 outputs data classified into the second group or the third group on the basis of its inference value so that the labeler can relabel the data. Here, the labeling guide unit 650 selects data that may help with (re) labeling the data so that the display unit 670 may output the selected data as samples together with the data.

First, the labeling guide unit 650 determines data of various classes that may correspond to input data so that the selected data may also be output as a sample. For example, the inference device 120 may infer one class with an absolutely high possibility or a plurality of classes with a marginal possibility difference from input data. In the latter case, the labeling guide unit 650 may select any data which has been classified into the classes with the marginal possibility difference as a sample. When data of various classes is output together with data classified into the second group or the third group, the labeler can be helped with (re) labeling of the input data.

In addition, the labeling guide unit 650 selects various types of data having the same feature as the input data as samples and also outputs the samples. As described above, data which has been classified into the second group or the third group on the basis of its inference value exists at the boundary between classes or corresponds to data of a new class. When such data is displayed, the labeling guide unit 650 may select other data classified into the same feature (group) as a sample and output the sample together. For example, when the display unit 670 outputs data determined as the second group, other data determined as the second group by the labeling guide unit 650 may be output together. Accordingly, in the case of (re) labeling data (currently) determined as the second group, the labeler may check data that has been determined as the second group and check with which class the data has been labeled.

The memory unit 660 stores the input data and the inference value received from the inference device 120 and stores the data selected by the data selection unit 640. In particular, the data selected by the data selection unit 640 is effective for retraining the trained model and may be an excellent resource for the trained model. Accordingly, the memory unit 660 may separately store the data selected by the data selection unit 640.

The display unit 670 outputs input data that is determined as the second group or the third group by the confidence calculation unit 620. The display unit 670 may output the input data so that the labeler can check the input data (so that the trained model may be retrained using the input data) and relabel the input data. In the case of outputting the data, the display unit 670 may assist the labeler in relabeling by outputting the data selected by the labeling guide unit 650 together.

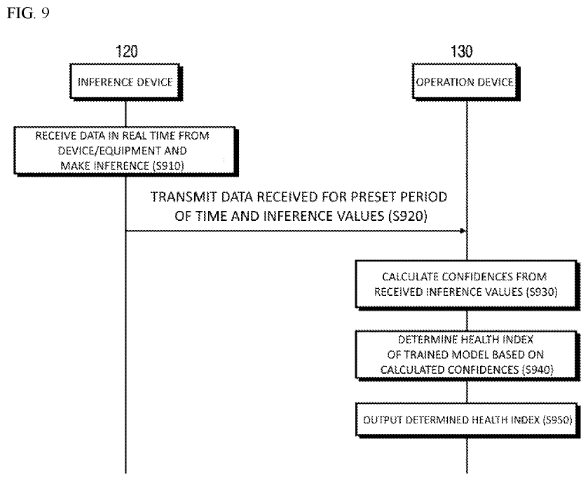

FIG. 9 is a timing chart illustrating a method in which the operation device according to the first embodiment of the present invention determines the health index of a trained model.

The inference device 120 receives data in real time from the apparatus/equipment 145 and makes inferences (S910).

The inference device 120 transmits input data which is input for a preset period of time and inference values (S920).

The operation device 130 calculates confidences from the received inference values (S930).

The operation device 130 determines the health index of a trained model of the inference device 120 on the basis of the calculated confidences (S940).

The operation device 130 outputs the determined health index (S950). The operation device 130 may directly output the determined health index through the display unit 670, and in some cases, transmit the health index to the training device 110 or the terminal (not shown) of the person concerned.

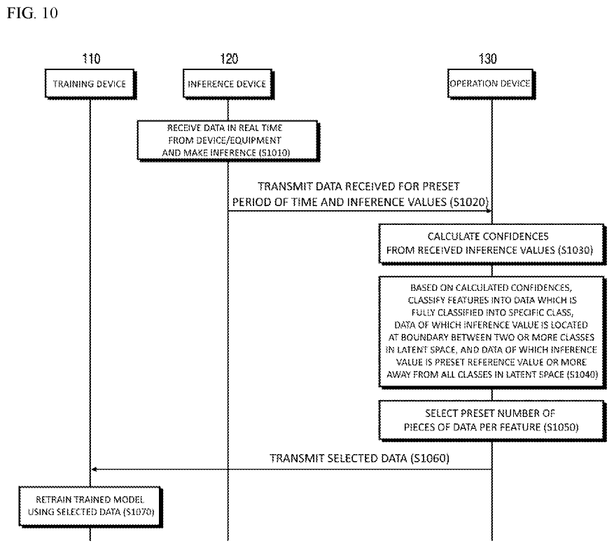

FIG. 10 is a timing chart illustrating a method in which the operation device according to the first embodiment of the present invention selects data for training or retraining.

The inference device 120 receives data in real time from the apparatus/equipment 145 and makes inferences (S1010).

The inference device 120 transmits input data which is input for a preset period of time and inference values (S1020).

The operation device 130 calculates confidences from the received inference values (S1030).

On the basis of the calculated confidences, the operation device 130 classifies features of the data into data that is fully classified into a specific class, data of which an inference value is located at the boundary between two or more classes in the latent space, and data of which an inference value is a preset reference value or more away from all the classes (S1040). The operation device 130 classifies each inference value into any one of the first group 810 to the third group 830 on the basis of the calculated confidence.

The operation device 130 selects a preset number of pieces of data per feature (S1050). The operation device 130 may select a preset ratio of the preset number of pieces of data per feature.

The operation device 130 transmits the selected data to the training device 110 (S1060).

The training device 110 retrains the trained model using the data selected by the operation device 130 (S1070).

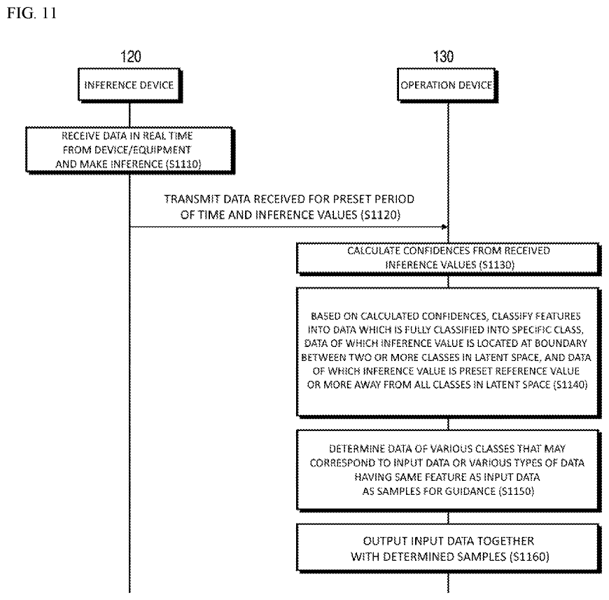

FIG. 11 is a timing chart illustrating a method in which the operation device according to the first embodiment of the present invention selects and outputs a sample together with an inference value of input data.

The inference device 120 receives data in real time from the apparatus/equipment 145 and makes inferences (S1110).

The inference device 120 transmits input data which is input for a preset period of time and inference values (S1120).

The operation device 130 calculates confidences from the received inference values (S1130).

On the basis of the calculated confidences, the operation device 130 classifies features of the data into data that is fully classified into a specific class, data of which an inference value is located at the boundary between two or more classes in the latent space, and data of which an inference value is a preset reference value or more away from all the classes in the latent space (S1140).

The operation device 130 determines data of various classes that may correspond to the input data or various types of data having the same feature as the input data as samples for guidance (S1150). The operation device 130 may select data of various classes that may correspond to the input data as samples for labeling guidance, select various pieces of data having the same feature as the input data as samples for labeling guidance, or select both as samples.

The operation device 130 outputs the input data together with the determined samples (S1160).

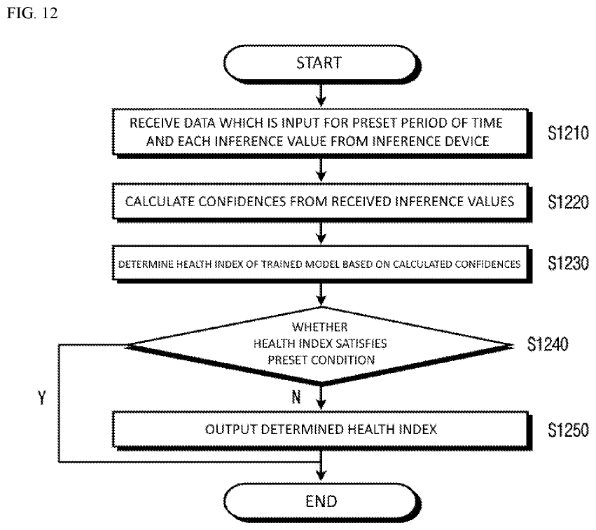

FIG. 12 is a flowchart illustrating a method in which the operation device according to the first embodiment of the present invention determines the health index of a trained model.

The communication unit 610 receives data which is input for a preset period of time and inference values from the inference device 120 (S1210).

The confidence calculation unit 620 calculates confidences from the received inference values (S1220).

The health index determination unit 630 determines the health index of a trained model on the basis of the calculated confidences (S1230).

The health index determination unit 630 determines whether the health index satisfies a preset condition (S1240). The preset condition may be whether there is a change in the number or confidences of inference values classified into the second group 820, whether there is a change in the number or confidences of inference values classified into the third group 830, whether there is a change in the radius rᵢ of each class, or whether there is a change in the coordinates of the center cᵢ of each class.

When the health index does not satisfy the preset condition, the display unit 670 outputs the determined health index (S1250). Also, the communication unit 610 may transmit the determined health index to the training device 110 or the terminal (not shown) of the person concerned.

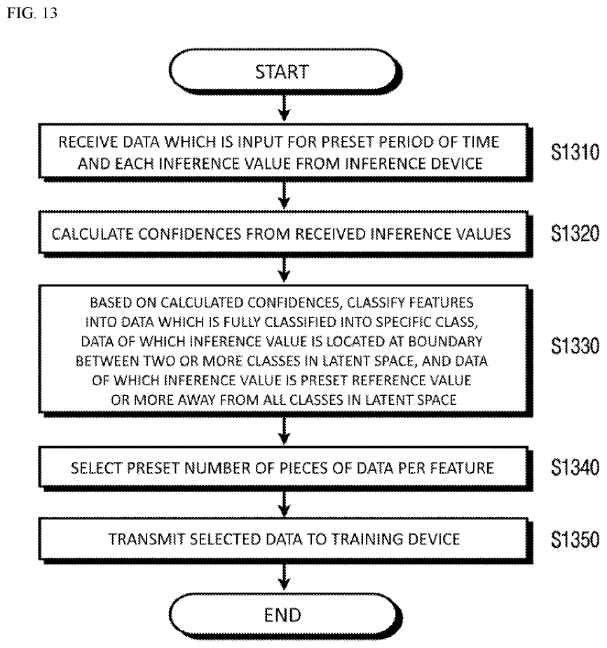

FIG. 13 is a flowchart illustrating a method in which the operation device according to the first embodiment of the present invention selects data for training or retraining.

The communication unit 610 receives data input for a preset period of time and inference values from the inference device 120 (S1310).

The confidence calculation unit 620 calculates confidences from the received inference values (S1320).

The confidence calculation unit 620 classifies features of the received input data into data that is fully classified into a specific class, data of which an inference value is located at the boundary between two or more classes in the latent space, and data of which an inference value is a preset reference value or more away from all the classes in the latent space (S1330).

The data selection unit 640 selects a preset number of pieces of data per feature (S1340).

The communication unit 610 transmits the selected data to the training device 110 (S1350).

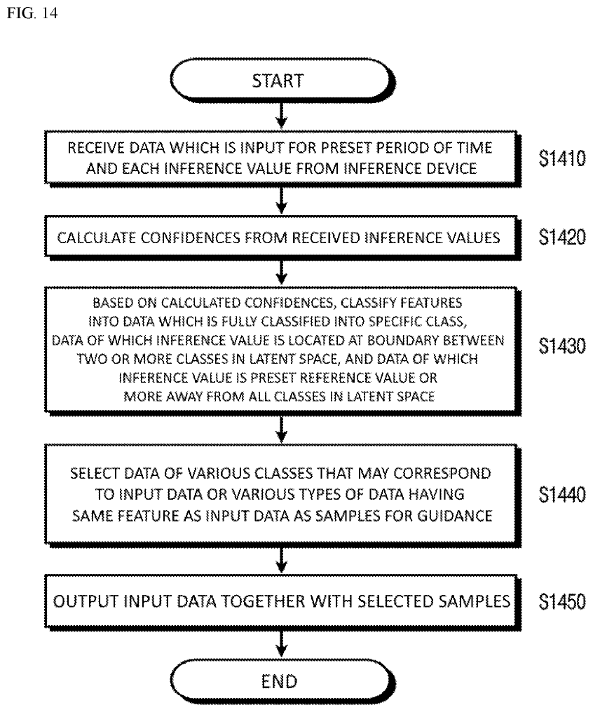

FIG. 14 is a flowchart illustrating a method in which the operation device according to the first embodiment of the present invention selects and outputs a sample together with an inference value of input data.

The communication unit 610 receives data input for a preset period of time and inference values from the inference device 120 (S1410).

The confidence calculation unit 620 calculates confidences from the received inference values (S1420).

The confidence calculation unit 620 classifies features of the received input data into data that is fully classified into a specific class, data of which an inference value is located at the boundary between two or more classes in the latent space, and data of which an inference value is a preset reference value or more away from all the classes in the latent space (S1430).

The labeling guide unit 650 selects data of various classes that may correspond to the input data or various types of data having the same feature as the input data as samples for guidance (S1440).

The display unit 670 outputs the input data and the selected samples together (S1450).

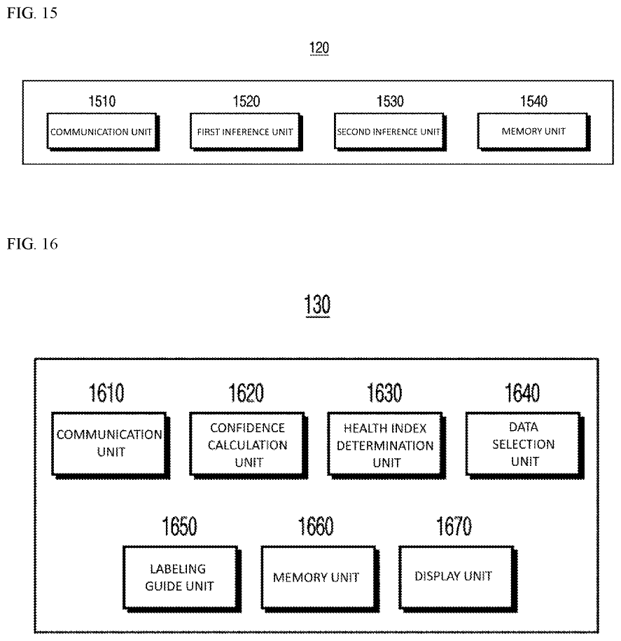

FIG. 15 is a diagram showing a configuration of an inference device according to a second embodiment of the present invention.

Referring to FIG. 15, the inference device 120 according to the second embodiment of the present invention includes a communication unit 1510, a first inference unit 1520, a second inference unit 1530, and a memory unit 1540.

The communication unit 1510 performs the same operations as the communication unit 510.

The first inference unit 1520 makes an inference using a received first trained model to classify the input data which is received in real time into a class. The first trained model is a model that is trained to minimize a cost value of a function which is proportional to the difference between an inference value and the result value (correct answer value) among the second cost functions. The first trained model does not cluster inference values for each class and only makes an inference to classify the input data into a specific class. Since the cost function is only defined as the difference between an inference value and the result value, the accuracy of an inference value may slightly increase compared to that of the trained model of the first embodiment.

The second inference unit 1530 makes an inference to classify input data which is input in real time into a class using a received second trained model. The second inference unit 1530 performs the same operations as the inference unit 520.

The memory unit 1540 stores the received trained models and input data and stores the inference values inferred by the inference units 1520 and 1530.

In this way, the inference device 120 makes a plurality of inferences using a plurality of trained models and thus can have the following merits. Since the first inference unit 1520 showing relatively high accuracy makes an inference for real-time class determination, it is possible to ensure inference accuracy. Also, the second inference unit 1530 makes an inference to determine the confidence of the operation device 130 or select data so that the operation device 130 can make a judgment on the basis of not only class inferences but also information on distance in the latent space and the like.

FIG. 16 is a diagram showing a configuration of an operation device according to the second embodiment of the present invention.

Referring to FIG. 16, the operation device 130 according to the second embodiment of the present invention includes a communication unit 1610, a confidence calculation unit 1620, a health index determination unit 1630, a data selection unit 1640, a labeling guide unit 1650, and a memory unit 1660. Further, the operation device 130 may include a display unit 1670.

The communication unit 1610 receives input data and an inference value from the first inference unit 1510 and the second inference unit 1520 of the inference device 120 and transmits a health index determination result or selected data to the training device 110 or the terminal (not shown) of the person concerned. Since both of the inference units 1510 and 1520 have the same input data, the communication unit 1610 receives inference values of both of the inference units 1510 and 1520 together with the input data.

The confidence calculation unit 1620 performs the same operations as the confidence calculation unit 620. However, since the confidence calculation unit 1620 requires information on distance in the latent space and the like to perform confidence calculation and the like, the confidence calculation unit 1620 uses the inference value inferred by the second inference unit 1530 rather than the inference value inferred by the first inference unit 1520 to perform confidence calculation and the like.

The health index determination unit 1630 also performs the same operations as the health index determination unit 630.

The data selection unit 1640 also performs the same operations as the data selection unit 640. Here, the data selection unit 1640 likewise uses the inference value inferred by the second inference unit 1530 rather than the inference value inferred by the first inference unit 1520 to select data.

The labeling guide unit 1650 also performs the same operations as the labeling guide unit 650. Here, in the case of selecting data of various classes that may correspond to the input data as samples, the labeling guide unit 1650 may use the inference value inferred by the first inference unit 1520 showing relatively high accuracy.

On the other hand, in the case of selecting various types of data having the same feature as the input data as samples, the labeling guide unit 1650 may use the inference value inferred by the second inference unit 1530.

The memory unit 1660 and the display unit 1670 also perform the same operations as the memory unit 660 and the display unit 670, respectively.

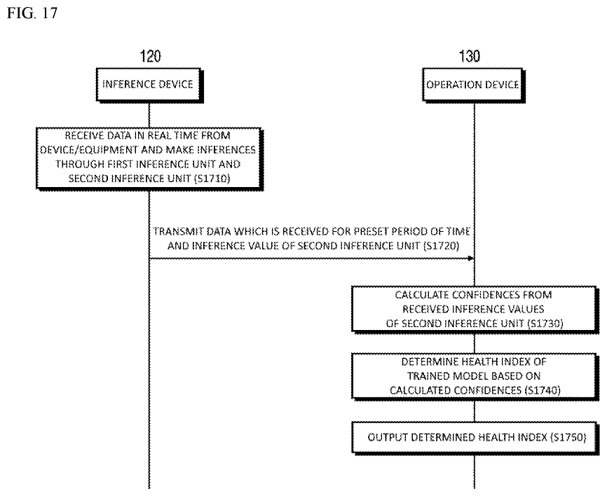

FIG. 17 is a timing chart illustrating a method in which the operation device according to the second embodiment of the present invention determines the health index of a trained model.

The inference device 120 receives data in real time from the apparatus/equipment 145, and each of the first inference unit 1520 and the second inference unit 1530 makes an inference (S1710).

The inference device 120 transmits input data which is input for a preset period of time and an inference value of the second inference unit 1530 (S1720).

The operation device 130 calculates confidences from the inference values received from the second inference unit 1530 (S1730).

The operation device 130 determines the health index of a trained model of the inference device 120 on the basis of the calculated confidences (S1740).

The operation device 130 outputs the determined health index (S1750). The operation device 130 may directly output the determined health index through the display unit 670, and in some cases, transmit the health index to the training device 110 or the terminal (not shown) of the person concerned.

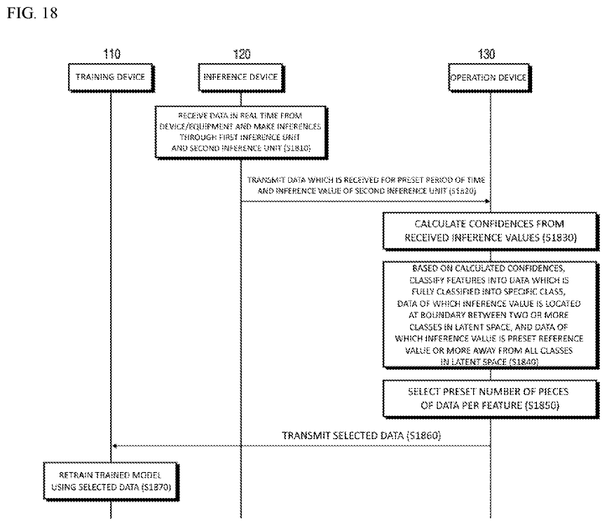

FIG. 18 is a timing chart illustrating a method in which the operation device according to the second embodiment of the present invention selects data for training or retraining.

The inference device 120 receives data in real time from the apparatus/equipment 145, and each of the first inference unit 1520 and the second inference unit 1530 makes an inference (S1810).

The inference device 120 transmits input data which is received for a preset period of time and an inference value of the second inference unit 1530 (S1820).

The operation device 130 calculates confidences from the inference values received from the second inference unit 1530 (S1830).

On the basis of the calculated confidences, the operation device 130 classifies features of the data into data that is fully classified into a specific class, data of which an inference value is located at the boundary between two or more classes in the latent space, and data of which an inference value is a preset reference value or more away from all the classes in the latent space (S1840).

The operation device 130 selects a preset number of pieces of data per feature (S1850). The operation device 130 may select a preset ratio of the preset number of pieces of data per feature.

The operation device 130 transmits the selected data to the training device 110 (S1860).

The training device 110 retrains a trained model using the data selected by the operation device 130 (S1870).

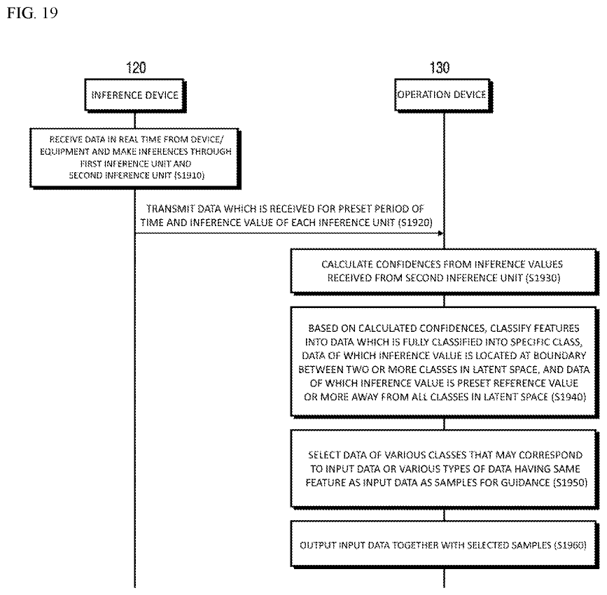

FIG. 19 is a timing chart illustrating a method in which the operation device according to the second embodiment of the present invention selects and outputs a sample together with an inference value of input data.

The inference device 120 receives data in real time from the apparatus/equipment 145, and each of the first inference unit 1520 and the second inference unit 1530 makes an inference (S1910).

The inference device 120 transmits input data which is received for a preset period of time and inference values of the second inference unit 1530 (S1920).

The operation device 130 calculates confidences from the inference values received from the second inference unit 1530 (S1930).

On the basis of the calculated confidences, the operation device 130 classifies features of the data into data that is fully classified into a specific class, data of which an inference value is located at the boundary between two or more classes in the latent space, and data of which an inference value is a preset reference value or more away from all the classes in the latent space (S1940).

The operation device 130 selects data of various classes that may correspond to the input data or various types of data having the same feature as the input data as samples for guidance (S1950).

The operation device 130 outputs the input data together with the selected samples (S1960).

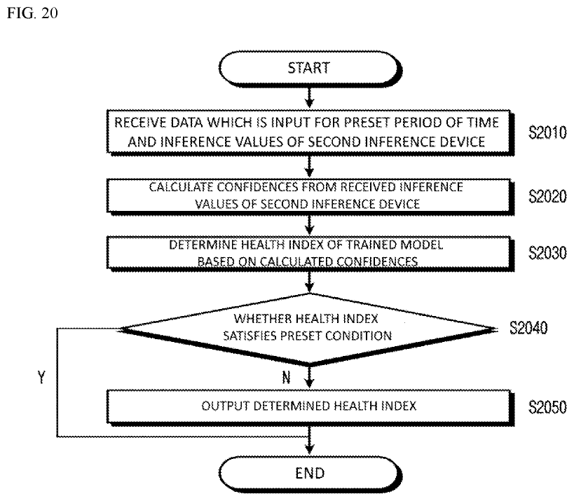

FIG. 20 is a flowchart illustrating a method in which the operation device according to the second embodiment of the present invention determines the health index of a trained model.

The communication unit 1610 receives data which is input for a preset period of time and inference values of the second inference unit 1530 from the second inference unit 1530 (S2010).

The confidence calculation unit 1620 calculates confidences from the inference values received from the second inference unit 1530 (S2020).

The health index determination unit 1630 determines the health index of a trained model on the basis of the calculated confidences (S2030).

The health index determination unit 1630 determines whether the health index satisfies a preset condition (S2040).

When the health index does not satisfy the preset condition, the display unit 1670 outputs the determined health index (S2050).

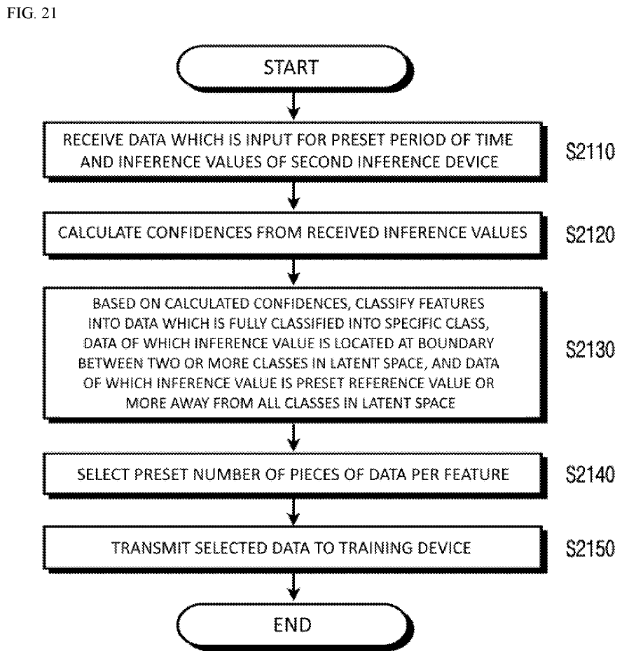

FIG. 21 is a flowchart illustrating a method in which the operation device according to the second embodiment of the present invention selects data for training or retraining.

The communication unit 1610 receives data which is input for a preset period of time and inference values of the second inference unit 1530 from the second inference unit 1530 (S2110).

The confidence calculation unit 1620 calculates confidences from the received inference values (S2120).

On the basis of the calculated confidences, the confidence calculation unit 1620 classifies features of the received input data into data that is fully classified into a specific class, data of which an inference value is located at the boundary between two or more classes in the latent space, and data of which an inference value is a preset reference value or more away from all the classes in the latent space (S2130).

The data selection unit 1640 selects a preset number of pieces of data per feature (S2140).

The communication unit 1610 transmits the selected data to the training device 110 (S2150).

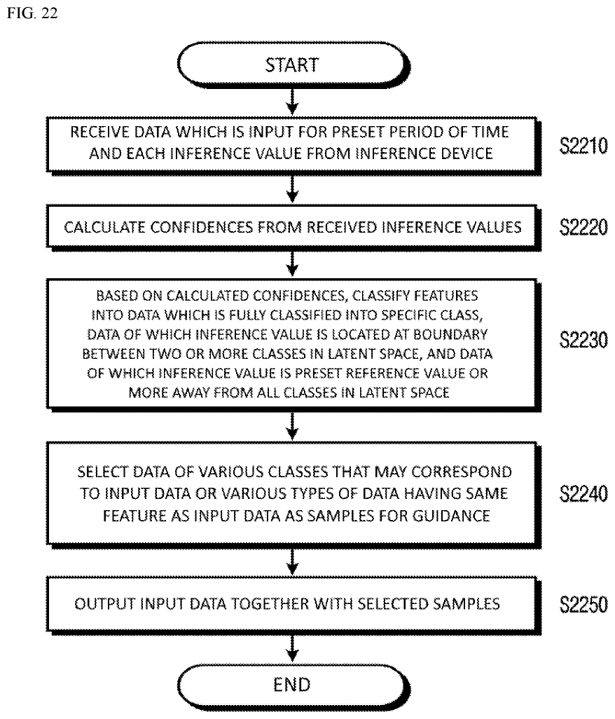

FIG. 22 is a flowchart illustrating a method in which the operation device according to the second embodiment of the present invention selects and outputs a sample together with an inference value of input data.

The communication unit 1610 receives data which is input for a preset period of time and each inference value from the inference unit 120 (S2210).

The confidence calculation unit 1620 calculates confidences from the received inference values (S2220).

On the basis of the calculated confidences, the confidence calculation unit 1620 classifies features of the received input data into data that is fully classified into a specific class, data of which an inference value is located at the boundary between two or more classes in the latent space, and data of which an inference value is a preset reference value or more away from all the classes in the latent space (S2230).

The labeling guide unit 1650 selects data of various classes that may correspond to the input data or various types of data having the same feature as the input data as samples for guidance (S2240).

The display unit 1670 outputs the input data and the selected samples together (S2250).

Although it is described in FIGS. 9 to 14 and FIGS. 17 to 24 that each process is sequentially executed, this merely illustrates technical spirit according to embodiments of the present invention. In other words, since those skilled in the technical field to which embodiments of the present invention pertain can apply each process in variously modified or altered forms by changing an order described in each drawing and executing the process or by parallelly executing one or more processes in the process, FIGS. 9 to 14 and FIGS. 17 to 24 are not limited to a time-series order.

Meanwhile, processes shown in FIGS. 9 to 14 and FIGS. 17 to 24 can be implemented as computer-readable code in a computer-readable recording medium. The computer-readable recording medium includes any type of recording device for storing data that may be read by a computer system. In other words, examples of the computer-readable recording medium include magnetic storage media (e.g., a read-only memory (ROM), a floppy disk, a hard disk drive, and the like) and storage media such as optical reading media (e.g., a compact disc (CD)-ROM, a digital versatile disc (DVD), and the like). Also, in the computer-readable recording medium, code that may be distributed over a computer system connected through a network and read by a computer in a distributed manner may be stored and executed.

The above description merely illustrates the technical spirit of the present embodiment, and various modifications and alterations will be possible without departing from the essential features of the present embodiment by those skilled in the technical field to which the present embodiment pertains. Accordingly, the present embodiment is intended to explain rather than limit the technical spirit of the present embodiment, and the scope of the technical spirit of the present embodiment is not limited by these embodiments. The protection scope of the present embodiment should be interpreted from the following claims, and all technical spirits within the scope equivalent thereto should be construed as falling within the scope of the present embodiment.

### CROSS-REFERENCE TO RELATED APPLICATION

*Under US Patent Code 119(a) (35 U.S.C § 119(a)), this application claims priority to and the benefit of Korean Patent Application Nos. 10-2021-0125303, 10-2021-0125304, 10-2021-0125306, and 10-2021-0125309, filed on Sep. 17, 2021, the disclosure of which is incorporated herein by reference in its entirety. In addition, this application claims priority for countries other than the US for the same reason as above, the disclosure of which is incorporated herein by reference.

1. A system for determining a health index of an artificial intelligence (AI) model which receives and classifies input data into any one of a plurality of classes, the system comprising:
a training device configured to generate a trained model for inferring a class of input data;
an inference device configured to receive the trained model generated by the training device, receive input data, and make an inference for classifying the input data into a class; and
an operation device configured to receive the input data and an inference value from the inference device, calculate a confidence of the input data from the inference value, and determine a health index of the trained model used by the inference device.

2. The system of claim 1, wherein the trained model is trained using labeled input data, an architecture, a cost function, and an optimization method.

3. The system of claim 2, wherein the trained model clusters data which is inferred as the same class in a latent space.

4. The system of claim 2, wherein the optimization method is a method of minimizing a cost value determined by the cost function.

5. The system of claim 2, wherein the confidence is a level of confidence that the input data will be classified into a specific class when the inference device makes an inference for classifying the input data into the specific class.

6. A method in which a system for determining a health index of an artificial intelligence (AI) model including a training device, an inference device, and an operation device receives and classifies input data into any one of a plurality of classes, the method comprising:
a generation process in which the training device generates a trained model for inferring a class of input data;
a first receiving process in which the inference device receives the trained model from the training device and receives input data;
an inference process in which the inference device makes an inference for classifying the input data into a class;
a second receiving process in which the operation device receives the input data and an inference value generated in real time from the inference device, and
a determination process in which the operation device calculates a confidence of the input data from the inference value and determines a health index of the trained model used by the inference device.

7. The method of claim 6, wherein the trained model is trained using labeled input data, an architecture, a cost function, and an optimization method.

8. The method of claim 7, wherein the trained model makes the inference for classifying the input data into a class and also clusters data which is inferred as the same class in a latent space.

9. The method of claim 7, wherein the optimization method is a method of minimizing a cost value determined by the cost function.

10. The method of claim 7, wherein the confidence is a level of confidence that the input data will be classified into a specific class when the inference device makes an inference for classifying the input data into the specific class.

11. A device for determining a health index of an artificial intelligence (AI) model which receives and classifies input data into any one of a plurality of classes, the device comprising:
a communication unit configured to receive input data which is input to the AI model and inference values with which the AI model classifies the input data into classes, and externally transmit a health index of the AI model;
a confidence calculation unit configured to calculate confidences using the received inference values;
a health index determination unit configured to determine the health index of the AI model according to whether the confidences calculated by the confidence calculation unit satisfy a preset condition; and
a memory unit configured to store the received input data and inference values.

12. The device of claim 11, wherein the confidence calculation unit classifies features of the input data from the calculated confidences.

13. The device of claim 12, wherein the features of the input data are classified into data of which an inference value is located within a radius of a specific class in a latent space and thus which is fully classified into a specific class only and is in a first group, data of which an inference value is located outside radii of all classes in the latent space but which is located relatively adjacent to two or more classes and is in a second group, and data of which an inference value is at a distance of a preset reference value or more from all the classes in the latent space and which is in a third group.

14. The device of claim 13, wherein the health index determination unit determines the health index of the AI model using a number or average confidence of inference values classified into the second group.

15. The device of claim 13, wherein the health index determination unit determines the health index of the AI model using a number or average confidence of inference values classified into the third group.

16. The device of claim 13, wherein the health index determination unit determines the health index of the AI model using a change in the radius of each class in the latent space.

17. The device of claim 13, wherein the health index determination unit determines the health index of the AI model using a change in coordinates of a center of each class in the latent space.
